SEQUENCE LISTING (8 sequences)

SEQ ID NO: 1 - DNA, 2064 bp, Saccharomyces sp.
atgatcagacagtccacactcaagaacttcgctattaagcgttgcttccaacaaatggcataccgtaacacacctgctatgagatcagtcgccctcgcgcaacgcttttatagttcatcttcccgttattatagtgcgtctccattgccagcttccaatagaccggaaccagccccaagtttcaacgtcgagccagtggcacaggtgcccgagccttctaaactggccaagagactacgcactgagcctgacatggacacatcctttgtcggcttaactggtggtcaaatattcaatgaaatgatgtctagacataatgtcgacactgtatttggctatccaggtggtgctatcttgcctgtgtatgatgccatccacaacagtgacaaatttactttcgtcttaccaagacacgaacaaggtgctggccacatggccgaaggttatgccagagcctctggtaaaccaggtgttgtcttggtgacctccggtccaggtgctactaacgtcattactccaatggcagatgccttcgcagacggtattccaatggtcgtttttacagggcaagtgccaactagtgccatcggtaccgatgccttccaagaagccgatgttgtcggtatttccagatcctgtaccaagtggaatgtcatggcaaaaaccgtggaagaattgccattgcgtatcaacgaagcttttgaaattgccactagtggtagaccaggtcccgtcttggttgacttaccaaaggacgtcacagcggccattttaagaaacccaattccaacaaaaactactttgccatcaaatgcgctaaaccaattaactagtcgtacacaggatgagttcgttttgcaaaacatcaatagagcagcagatttgattaatttggcaaagaagcctgttttatacgtcggtgcaggtattttaaaccatgctgatggtccaagattactaaaagagttaagtgaccgtgcacaaatacccgttacaactactttgcaaggtttgggtgctttcgaccaagacgatccaaaatcccttgatatgttaggtatgcatggctgtgccactgctaacttggcggtacaaaatgcggatttgctaattgctgtaggtgctagattcgatgatcgtgtcaccttgaatatcgctaaatttgcaccagaggcccgtcgtgctgctgctgaaggcagaggtggtattatccattttgaagttagccccaaaaacataaacaaggtcgtggagactcaaatagcggtggaaggtgacgctactgggaatttggataaaatgatgccaaaaatctttccagtaaaggagagatcagagtggtttggtcaaataaacgaatggaagaagaaatacccatacgcttacatgatggaaactccaggatctaaaataaaaccacaaacagtcataacaaaactatccaagatcgccaacgacacaggaagacatgttatagtcacaaccggtgtcgggcaacatcaaatgtgggctgcccaacactggacatggaaaaccccacgtaccttcatcacatcaggtggtttaggtacaatgggttacggtcttccagccgccatcggcgctcaagtcgctaaaccagattctttggttattgacattgatggtgatgcatctttcaacatgaccttgaccgaattgagttctgccgtccaagctggtactccagtgaagatcttggttttgaacaatgaagaacaaggtatggttactcaatggcaatcactattttacgagaatcgttattctcacacacatcagttgaaccctgatttcataaaactagccgacgctatgggtctaaagggtttaagagtcaaaaagcaagaagaattagatgctaaattgaaagaatttgtttccacaaagggccctgtcttgcttgaagtggaagttgacaaaaaagttcctgttttgccaatggttccagcaggcaagggtctagatgagttcatatactttgatccagaggttgaaagacaacaaaatgaattacgtcacaagcgtaccaatggtaagcattga

SEQ ID NO: 2 - protein, 687 aa, Saccharomyces sp.
MIRQSTLKNFAIKRCFQQMAYRNTPAMRSVALAQRFYSSSSRYYSASPLPASNRPEPAPSFNVEPVAQVPEPSKLAKRLRTEPDMDTSFVGLTGGQIFNEMMSRHNVDTVFGYPGGAILPVYDAIHNSDKFTFVLPRHEQGAGHMAEGYARASGKPGVVLVTSGPGATNVITPMADAFADGIPMVVFTGQVPTSAIGTDAFQEADVVGISRSCTKWNVMAKTVEELPLRINEAFEIATSGRPGPVLVDLPKDVTAAILRNPIPTKTTLPSNALNQLTSRTQDEFVLQNINRAADLINLAKKPVLYVGAGILNHADGPRLLKELSDRAQIPVTTTLQGLGAFDQDDPKSLDMLGMHGCATANLAVQNADLLIAVGARFDDRVTLNIAKFAPEARRAAAEGRGGIIHFEVSPKNINKVVETQIAVEGDATGNLDKMMPKIFPVKERSEWFGQINEWKKKYPYAYMMETPGSKIKPQTVITKLSKIANDTGRHVIVTTGVGQHQMWAAQHWTWKTPRTFITSGGLGTMGYGLPAAIGAQVAKPDSLVIDIDGDASFNMTLTELSSAVQAGTPVKILVLNNEEQGMVTQWQSLFYENRYSHTHQLNPDFIKLADAMGLKGLRVKKQEELDAKLKEFVSTKGPVLLEVEVDKKVPVLPMVPAGKGLDEFIYFDPEVERQQNELRHKRTNGKH

SEQ ID NO: 3 - DNA, 24 bp, Artificial
  Primer
gagctctagaaatccactcgccaa

SEQ ID NO: 4 - DNA, 26 bp, Artificial
  Primer
ggatcctcaatgcttaccattggtac

SEQ ID NO: 5 - DNA, 120 bp, Artificial
  Primer
atgatcagacaatctacgctaaaaaacttcgctattaagcgttgctttcaacatatagcataccgcaacacacctgccatgagatcagtagctctcgcgcccttgacagtcttgacgtgc

SEQ ID NO: 6 - DNA, 120 bp, Artificial
  Primer
ttcagtgcttaccgcctgtacgcttatgacgtaattcagtctgttgtctttcaacttctgggtcaaaatttatgaactcgtctagaccgctaccacctgccgcacttaacttcgcatctg

SEQ ID NO: 7 - DNA, 80 bp, Artificial
  Primer
attgcattaacatagtctatacgcataggagaaaacacacaatagaaatccactcgccaaccttgacagtcttgacgtgc

SEQ ID NO: 8 - DNA, 80 bp, Artificial
  Primer
tataagcatggttaaataataattaagtctaaatttttaatggagaatgtgtttcagactcgcacttaacttcgcatctg

(19) United States
(12) Patent Application Publication
(10) Pub. No.: US 2009/0087515 A1
(43) Pub. Date: Apr. 2, 2009

(54) GENE ENCODING ACETOLACTATE SYNTHASE AND USE THEREOF

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: Suntory Limited, Osaka (JP)

(21) Appl. No.: 12/279,001

(22) PCT Filed: Nov. 30, 2006
(86) PCT No.: PCT/JP2006/324404

§ 371 Date: Aug. 11, 2008

(30) Foreign Application Priority Data
Feb. 23, 2006 (JP) 2006-047558

(51) Int. Cl. C12G 1/00; C07K 14/395; C12N 15/63; C12N 1/19; C12C 11/00; C12Q 1/68

(52) U.S. Cl. 426/11; 536/23.74; 530/350; 435/320.1; 435/254.21; 426/592; 435/6

(57) ABSTRACT

The present invention relates to an acetolactate synthase gene and use thereof, in particular, a brewery yeast for producing alcoholic beverages with superior flavor, alcoholic beverages produced with said yeast, and a method for producing said beverages. More particularly, the present invention relates to a yeast, whose capability of producing vicinal diketones, especially diacetyl, that are responsible for off-flavors in products, is reduced by repressing expression level of ILV2 gene encoding an acetolactate synthase (Ilv2p), especially non-ScILV2 gene specific to a lager brewing yeast, and to a method for producing alcoholic beverages with said yeast.

### TECHNICAL FIELD

The present invention relates to a gene encoding an acetolactate synthase and use thereof, in particular, a brewery yeast for producing alcoholic beverages with superior flavor, alcoholic beverages produced with said yeast, and a method for producing said beverages. More particularly, the present invention relates to a yeast, whose amount of production of vicinal diketone(s), especially diacetyl, that are responsible for off-flavors in products, is reduced by repressing expression level of ILV2 gene encoding an acetolactate synthase of a brewery yeast, especially non-ScILV2 gene specific to a lager brewing yeast, and to a method for producing alcoholic beverages with said yeast.

### BACKGROUND ART

Flavor of Diacetyl, hereinafter also referred to as “DA”, is a representative off-flavor in brewed alcoholic beverages such as beer, sake and wine and so on among flavoring substances of alcoholic beverages. DA flavor, which is also referred to as “butter flavor” or “sweaty flavor” in beer, “tsuwari-ka”, which means a nauseating flavor, in sake, occurs when vicinal diketone(s), hereinafter also referred to as “VDK”, mainly DA, are present above certain threshold levels in products. The threshold level is said to be 0.1 ppm (parts per million) in beer (Journal of the Institute of Brewing, 76, 486 (1979)).

VDK in alcoholic beverages can be broadly divided into DA and 2,3-pentanedione, herein after referred to as “PD”. DA and PD are formed by non-enzymatic reactions, which yeasts are not involved in, of α-acetolactic-acid and α-acetohydroxybutyric-acid as precursors which are intermediate products in biosynthesis of valine and isoleucine, respectively.

According to these information, VDKs (i.e., DA and PD) and their precursors α-acetohydroxy-acids (i.e., α-acetolactic-acid and α-acetohydroxybutyric-acid) are thought to be the compounds which can impart DA flavors to products. Accordingly, breeding of yeasts which steadily reduces these compounds makes manufacturing control of alcoholic beverages easy as well as expands capability of developing new products.

A method for suppressing production of DA by using rice-malt-yeast culture containing low level of pyruvic acid which is a precursor of acetohydroxy-acids in production of sake is reported as a method for controlling DA flavor in, for example, Japanese Patent Application Laid-Open No. 2001-204457. In addition, there are ILV2 and ILV6 as genes encoding a yeast acetolactate synthase, which is a enzyme converting pyruvic acid or α-oxobutyric acid to α-acetolactic-acid or α-acetohydroxybutyric-acid, respectively. It is known that ILV2 codes for active subunit, and ILV6 codes for regulatory subunit. Mutating or disrupting the ILV2 results in decrease of synthesis of precursors (α-acetohydroxy-acids) by suppressing activity of the enzyme mentioned above, and as a result, concentration of DA is reduced is reported in Journal of Basic Microbiology, 28 (3), 175-183 (1988). Regarding Ilv6p, which is a regulatory subunit, it is not appeared if it affects production of DA, although acetolactate synthase activity is analyzed enzymologically in, for example, Biochemistry 38 (16), 5222-31 (1999).

It is also reported that production of VDKs are reduced in valine, leucine and isoleucine auxotrophic yeast in beer production. However, the yeast has not come into practical use since auxotrophic strains tend to show retarded growth/fermentation. Japanese Patent Application Laid-Open NO. 2002-291465 discloses a method obtaining variant strains sensitive to analogues' of the branched amino acids described above, and selecting DA low accumulating strains from the variant strains. Genetically engineered yeasts derived from laboratory-designed yeasts whose amount of expression of ILV5 gene is regulated is reported in Journal of American Society of Brewing Chemists, Proceeding, 81-84 (1987), and also genetically engineered yeast whose amount of expression of ILV3 gene is regulated is reported in European Brewery Convention, Proceedings, of the 21st EBC congress, Madrid, 553-560 (1987). The enzymatic activity of the acetohydroxy-acid reductoisomerase encoded by ILV5 gene is increased 5 to 7-fold, and the amount of production of VDKs are reduced to about 40% in the case.

Besides, the enzymatic activity of the dihydroxy-acid dehydratase encoded by ILV3 is increased 5 to 6-fold. On the contrary, no significant reduction of the amount of production of VDKs was observed. However, any influence on practical beer brewing is analyzed in the two reports described above where synthetic media are used. On the other hand, Villa et al. reported in Journal of American Society of Brewing Chemists, 53; 49-53 (1995), that plasmid amplification of the gene products of ILV5, ILV3 or tandem ILV5+ILV3 in brewer's yeast resulted in VDK decreases of 70, 40 and 60% respectively, when compared to that of normal brewer's yeast on practical beer brewing.

Also, Dulieu et al. proposed a method converting α-acetolactic-acid, which served as a precursor of DA, rapidly to acetoin using α-acetolactate decarboxylase in European Brewery Convention, Proceedings of the 26th EBC congress, Maastricht, 455-460 (1997). However, α-acetolactate decarboxylase is an enzyme prepared only by utilizing recombinant DNA technology, and thus use of the enzyme is not acceptable due to consumers' negative images in Japan. Genetically engineered yeasts using DNA strands encoding α-acetolactate decarboxylase are reported in both Japanese Patent Application Laid-Open Nos. H2-265488 and H07-171.

### DISCLOSURE OF INVENTION

Under the circumstances described above, there were demands for developing a method for producing alcoholic beverages with superior flavor by breeding a yeast with low VDK-producing ability utilizing a gene encoding a protein capable of reducing the smell of VDKs (vicinal diketones), especially DA (diacetyl), and the protein.

To solve the problems described above, the present inventors made extensive studies, and as a result succeeded in identifying and isolating a gene encoding an acetolactate synthase.

Thus, the present invention relates to a novel acetolactate synthase gene existing specifically in a lager brewing yeast, to a protein encoded by said gene, to a transformed yeast in which the expression of said gene is controlled, to a method for controlling the level of VDKs, especially the level of DA, in a product by using a yeast in which the expression of said gene is controlled. More specifically, the present invention provides the following polynucleotides, a vector or DNA fragments comprising said polynucleotide, a transformed yeast introduced with said vector or said DNA fragments, a method for producing alcoholic beverages by using said transformed yeast, and the like:

(1) A polynucleotide selected from the group consisting of:

(a) a polynucleotide comprising a polynucleotide consisting of the nucleotide sequence of SEQ ID NO:1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 with one or more amino acids thereof being deleted, substituted, inserted and/or added, and having an acetolactate synthase activity;

(d) a polynucleotide comprising a polynucleotide encoding a protein having an amino acid sequence having 60% or higher identity with the amino acid sequence of SEQ ID NO:2, and having an acetolactate synthase activity;

(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having an acetolactate synthase activity; and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having an acetolactate synthase activity.

(2) The polynucleotide of (1) above selected from the group consisting of

(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has an acetolactate synthase activity;

(h) a polynucleotide encoding a protein having 90% or higher identity with the amino acid sequence of SEQ ID NO: 2, and having an acetolactate synthase activity; and

(i) a polynucleotide which hybridizes to SEQ ID NO: 1 or which hybridizes to a nucleotide sequence complementary to the nucleotide sequence of SEQ ID NO: 1 under stringent conditions, and which encodes a protein having an acetolactate synthase activity.

(3) The polynucleotide of (1) above comprising a polynucleotide consisting of SEQ ID NO: 1.

(4) The polynucleotide of (1) above comprising a polynucleotide encoding a protein consisting of SEQ ID NO: 2.

(5) The polynucleotide of any one of (1) to (4) above, wherein the polynucleotide is DNA.

(6) A polynucleotide selected from the group consisting of

(j) a polynucleotide encoding RNA of a nucleotide sequence complementary to a transcript of the polynucleotide (DNA) according to (5) above;

(k) a polynucleotide encoding RNA that represses the expression of the polynucleotide (DNA) according to (5) above through RNAi effect;

(l) a polynucleotide encoding RNA having an activity of specifically cleaving a transcript of the polynucleotide (DNA) according to (5) above; and

(m) a polynucleotide encoding RNA that represses expression of the polynucleotide (DNA) according to (5) above through co-suppression effect.

(7) A protein encoded by the polynucleotide of any one of (1) to (5) above.

(8) A vector comprising the polynucleotide of any one of (1) to (5) above.

(8a) The vector of (8) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(9) A vector comprising the polynucleotide of (6) above.

(10) A yeast, wherein the vector of (8) or (9) above is introduced.

(11) The yeast of (10) above, wherein a total vicinal diketone-producing capability or total diacetyl-producing capability is reduced by introducing the vector of (8) or (9) above.

(12) The yeast of (11) above, wherein a total vicinal diketone-producing capability or total diacetyl-producing capability is reduced by decreasing an expression level of the protein of (7) above.

(13) A yeast, wherein an expression level of the polynucleotide of (5) above is reduced by introducing the vector of (8) or (9) above, or by disrupting a gene related to the polynucleotide (DNA) of (5) above.

(14) A method for producing an alcoholic beverage comprising culturing the yeast of any one of (10) to (13) above.

(15) The method for producing an alcoholic beverage of (14) above, wherein the brewed alcoholic beverage is a malt beverage.

(16) The method for producing an alcoholic beverage of (14) above, wherein the brewed alcoholic beverage is wine.

(17) An alcoholic beverage produced by the method of any one of (14) to (16) above.

(18) A method for assessing a test yeast for its total vicinal diketone-producing capability or total diacetyl-producing capability, comprising using a primer or a probe designed based on a nucleotide sequence of an acetolactate synthase gene having the nucleotide sequence of SEQ ID NO: 1.

(18a) A method for selecting a yeast having a low total vicinal diketone-producing capability or total diacetyl-producing capability by using the method described in (18) above.

(18b) A method for producing an alcoholic beverage (for example, beer) by using the yeast selected with the method in (18a) above.

(19) A method for assessing a test yeast for its total vicinal diketone or total diacetyl-producing capability, comprising: culturing a test yeast; and measuring an expression level of an acetolactate synthase gene having the nucleotide sequence of SEQ ID NO: 1.

(19a) A method for selecting a yeast, which comprises assessing a test yeast by the method described in (19) above and selecting a yeast having a low expression level of the acetolactate synthase gene.

(19b) A method for producing an alcoholic beverage (for example, beer) by using the yeast selected with the method in (19a) above.

(20) A method for selecting a yeast, comprising: culturing test yeasts; quantifying the protein according to (7) or measuring an expression level of an acetolactate synthase gene having the nucleotide sequence of SEQ ID NO: 1; and selecting a test yeast having said protein amount or said gene expression level according to a target total vicinal diketone-producing capability or total diacetyl-producing capability.

(21) The method for selecting a yeast according to (20) above, comprising: culturing a reference yeast and test yeasts; measuring an expression level of an acetolactate synthase gene having the nucleotide sequence of SEQ ID NO: 1 in each yeast; and selecting a test yeast having the gene expressed lower than that in the reference yeast.

(22) The method for selecting a yeast according to (20) above, comprising: culturing a reference yeast and test yeasts; quantifying the protein according to (7) above in each yeast; and selecting a test yeast having said protein for a smaller amount than that in the reference yeast. That is, the method for selecting a yeast of (20) above, comprising: culturing plural yeasts; quantifying the protein of (7) above in each yeast; and selecting a yeast having a smaller amount of the protein among them.

(23) A method for producing an alcoholic beverage comprising: conducting fermentation for producing an alcoholic beverage using the yeast according to any one of (10) to (13) above or a yeast selected by the method according to any one of (20) to (22) above; and adjusting the total vicinal diketone-producing capability or total diacetyl-producing capability.

According to the method for producing alcoholic beverages of the present invention, because it is possible to reduce the production amount of VDKs (e.g., diacetyl (DA), 2,3-pentanedione (PD), etc.) or precursors thereof (e.g., α-acetohydroxy-acids, etc.), especially DA or precursors thereof (e.g., α-acetolactate, etc.), which are responsible for off-flavors in products, alcoholic beverages with superior flavor can be readily produced.

### BRIEF DESCRIPTION OF DRAWINGS

FIG. 1 shows the cell growth with time upon beer fermentation test. The horizontal axis represents fermentation time while the vertical axis represents optical density at 660 nm (OD660).

FIG. 2 shows the extract consumption with time upon beer fermentation test. The horizontal axis represents fermentation time while the vertical axis represents apparent extract concentration (w/w %).

FIG. 3 shows the expression behavior of non-ScILV2 gene in yeasts upon beer fermentation test. The horizontal axis represents fermentation time while the vertical axis represents the brightness of detected signal.

FIG. 4 shows the result of complementation test of nonScILV2 using ILV2 gene-disrupted strain.

a) This figure shows that ILV2 gene disruption causes auxotrophy for valine, leucine and isoleucine. The parent strain was S. cerevisiae X2180-1A. The strains were cultured for 3 days at 30° C. on SC (-Leu, Ile, Val) plate medium.

b) This figure shows that introduction of nonScILV2 gene into ILV2 gene-disrupted strain makes the strain non-auxotrophic. The parent strain was X2180-1A (ilv2::nat1). The strains were cultured for 3 days at 30° C. on SC (-Leu, Ile, Val) plate medium containing 300 mg/L geneticin.

### BEST MODES FOR CARRYING OUT TIE INVENTION

The present inventors isolated and identified non-ScILV2 gene encoding an acetolactate synthase unique to lager brewing yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2.

Meanwhile, VDK and α-acetohydroxy-acid, which is a precursor of the VDK, are sometimes referred to as “total vicinal diketone(s).” Further, DA and α-acetolactate, which is a precursor of the DA, are sometimes collectively referred to as “total diacetyl(s).”

### 1. Polynucleotide of the Invention

First of all, the present invention provides (a) a polynucleotide comprising a polynucleotide of the nucleotide sequence of SEQ ID NO: 1; and (b) a polynucleotide comprising a polynucleotide encoding a protein of the amino acid sequence of SEQ ID NO:2. The polynucleotide can be DNA or RNA.

The target polynucleotide of the present invention is not limited to the polynucleotide encoding an acetolactate synthase derived from lager brewing yeast and may include other polynucleotides encoding proteins having equivalent functions to said protein. Proteins with equivalent functions include, for example, (c) a protein of an amino acid sequence of SEQ ID NO: 2 with one or more amino acids thereof being deleted, substituted, inserted and/or added and having an acetolactate synthase activity.

Such proteins include a protein consisting of an amino acid sequence of SEQ ID NO: 2 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having an acetolactate synthase activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 0.94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2, and having an acetolactate synthase activity. In general, the percentage identity is preferably higher.

The acetolactate synthase activity can be assessed, for example by, a method of Pang et al. (Biochemistry, 38, 5222-5231, 1999).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 under stringent conditions and which encodes a protein having an acetolactate synthase activity; and (f) a polynucleotide comprising a polynucleotide which hybridizes to a polynucleotide complementary to a nucleotide sequence of encoding a protein of SEQ ID NO: 2 under stringent conditions, and which encodes a protein having an acetolactate synthase activity.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or polynucleotide encoding the amino acid sequence of SEQ ID NO: 2 as a probe. The hybridization method may be a method described, for example, in Molecular Cloning 3rd Ed., Current Protocols in Molecular Biology, John Wiley & Sons 1987-1997.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

The polynucleotide of the present invention includes (j) a polynucleotide encoding RNA having a nucleotide sequence complementary to a transcript of the polynucleotide (DNA) according to (5) above; (k) a polynucleotide encoding RNA that represses the expression of the polynucleotide (DNA) according to (5) above through RNAi effect; (l) a polynucleotide encoding RNA having an activity of specifically cleaving a transcript of the polynucleotide (DNA) according to (5) above; and (m) a polynucleotide encoding RNA that represses expression of the polynucleotide (DNA) according to (5) above through co-suppression effect. These polynucleotides may be incorporated into a vector, which can be introduced into a cell for transformation to repress the expression of the polynucleotides (DNA) of (a) to (i) above. Thus, these polynucleotides may suitably be used when repression of the expression of the above polynucleotide (DNA) is preferable.

The phrase “polynucleotide encoding RNA having a nucleotide sequence complementary to the transcript of DNA” as used herein refers to so-called antisense DNA. Antisense technique is known as a method for repressing expression of a particular endogenous gene, and is described in various publications (see e.g., Hirajima and Inoue: New Biochemistry Experiment Course 2 Nucleic Acids IV Gene Replication and Expression (Japanese Biochemical Society Ed., Tokyo Kagaku Dozin Co., Ltd.) pp. 319-347, 1993). The sequence of antisense DNA is preferably complementary to all or part of the endogenous gene, but may not be completely complementary as long as it can effectively repress the expression of the gene. The transcribed RNA has preferably 90% or higher, and more preferably 95% or higher complementarity to the transcript of the target gene. The length of the antisense DNA is at least 15 bases or more, preferably 100 bases or more, and more preferably 500 bases or more.

The phrase “polynucleotide encoding RNA that represses DNA expression through RNAi effect” as used herein refers to a polynucleotide for repressing expression of an endogenous gene through RNA interference (RNAi). The term “RNAi” refers to a phenomenon where when double-stranded RNA having a sequence identical or similar to the target gene sequence is introduced into a cell, the expressions of both the introduced foreign gene and the target endogenous gene are repressed. RNA as used herein includes, for example, double-stranded RNA that causes RNA interference of 21 to 25 base length, for example, dsRNA (double strand RNA), siRNA (small interfering RNA) or shRNA (short hairpin RNA). Such RNA may be locally delivered to a desired site with a delivery system such as liposome, or a vector that generates the double-stranded RNA described above may be used for local expression thereof. Methods for producing or using such double-stranded RNA (dsRNA, siRNA or shRNA) are known from many publications (see, e.g., Japanese National Phase PCT Laid-open Patent Publication No. 2002-516062; US 2002/086356A; Nature Genetics, 24(2), 180-183, 2000 February; Genesis, 26(4), 240-244, 2000 April; Nature, 407:6802, 319-20, 2002 September 21; Genes & Dev., Vol. 16, (8), 948-958, 2002 Apr. 15; Proc. Natl. Acad. Sci. USA, 99(8), 5515-5520, 2002 Apr. 16; Science, 296(5567), 550-553, 2002 Apr. 19; Proc Natl. Acad. Sci. USA, 99:9, 6047-6052, 2002 Apr. 30; Nature Biotechnology, Vol. 20 (5), 497-500, 2002 May; Nature Biotechnology, Vol. 20(5), 500-505, 2002 May; Nucleic Acids Res., 30:10, e46, 2002 May 15).

The phrase “polynucleotide encoding RNA having an activity of specifically cleaving transcript of DNA” as used herein generally refers to a ribozyme. Ribozyme is an RNA molecule with a catalytic activity that cleaves a transcript of a target DNA and inhibits the function of that gene. Design of ribozymes can be found in various known publications (see, e.g., FEBS Lett. 228: 228, 1988; FEBS Lett. 239: 285, 1988; Nucl. Acids. Res. 17: 7059, 1989; Nature 323: 349, 1986; Nucl. Acids. Res. 19: 6751, 1991; Protein Eng 3: 733, 1990; Nucl. Acids Res. 19: 3875, 1991; Nucl. Acids Res. 19: 5125, 1991; Biochem Biophys Res Commun 186: 1271, 1992). In addition, the phrase “polynucleotide encoding RNA that represses DNA expression through co-suppression effect” refers to a nucleotide that inhibits functions of target DNA by “co-suppression”.

The term “co-suppression” as used herein, refers to a phenomenon where when a gene having a sequence identical or similar to a target endogenous gene is transformed into a cell, the expressions of both the introduced foreign gene and the target endogenous gene are repressed. Design of polynucleotides having a co-suppression effect can also be found in various publications (see, e.g., Smyth D R: Curr. Biol. 7: R793, 1997, Martienssen R: Curr. Biol. 6: 810, 1996).

### 2. Protein of the Present Invention

The present invention also provides proteins encoded by any of the polynucleotides (a) to (i) above. A preferred protein of the present invention comprises an amino acid sequence of SEQ ID NO:2 with one or several amino acids thereof being deleted, substituted, inserted and/or added, and has an acetolactate synthase activity.

Such protein includes those having an amino acid sequence of SEQ ID NO: 2 with amino acid residues thereof of the number mentioned above being deleted, substituted, inserted and/or added and having an acetolactate synthase activity. In addition, such protein includes those having homology as described above with the amino acid sequence of SEQ ID NO: 2 and having an acetolactate synthase activity.

Such proteins may be obtained by employing site-directed mutation described, for example, in Molecular Cloning 3rd Ed., Current Protocols in Molecular Biology, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides (such as DNA) described in (a) to (i) above. Generally, the vector of the present invention comprises an expression cassette including as components (x) a promoter that can transcribe in a yeast cell; (y) a polynucleotide (such as DNA) described in any of (a) to (i) above that is linked to the promoter in sense or antisense direction; and (z) a signal that functions in the yeast with respect to transcription termination and polyadenylation of RNA molecule. Further, the polynucleotides may be introduced into vectors which comprises the polynucleotide of the (j) to (m) above such that the polynucleotide is to be expressed. According to the present invention, the target gene (DNA) may be disrupted to repress the expression of the DNA described above or the expression of the protein described above. A gene may be disrupted by adding or deleting one or more bases to or from a region involved in expression of the gene product in the target gene, for example, a coding region or a promoter region, or by deleting these regions entirely. Such disruption of gene may be found in known publications (see, e.g., Proc. Natl. Acad. Sci. USA, 76, 4951 (1979), Methods in Enzymology, 101, 202 (1983), Japanese Patent Application Laid-Open No. 6-253826).

A vector introduced in the yeast may be any of a multicopy type (YEp type), a single copy type (YCp type), or a chromosome integration type (YIp type). For example, YEp24 (J. R. Broach et al., Experimental Manipulation of Gene Expression, Academic Press, New York, 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they have no influence on the concentration of constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yeasts such as genus Saccharomyces may be used. According to the present invention, a lager brewing yeast, for example, Saccharomyces pastorianus W34/70, Saccharomyces carlsbergensis NCYC453 or NCYC456, or Saccharomyces cerevisiae NBRC1951, NBRC1952, NBRC1953 or NBRC1954 may be used. In addition, whiskey yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), Methods in Yeast Genetics, 2000 Edition: A Cold Spring Harbor Laboratory Course Manual.

More specifically, a host yeast is cultured in a standard yeast nutrition medium (e.g., YEPD medium (Genetic Engineering Vol. 1, Plenum Press, New York, 117 (1979)), etc.) such that OD600 nm will be 1 to 6. This culture yeast is collected by centrifugation, washed and pre-treated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in Molecular Cloning 3rd Ed., and Methods in Yeast Genetics, A Laboratory Manual (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to reduce the level of VDKs, especially DA, of desired alcoholic beverages, and produce alcoholic beverages having enhanced flavor. Specifically, desired kind of alcoholic beverages with reduced level of VDKs, especially DA, can be produced by reducing production amount of VDKs, especially production amount of DA using yeasts into which the vector of the present invention was introduced as described above, yeasts in which expression of the polynucleotide (DNA) of the present invention described above was suppressed or yeasts selected by the yeast assessment method of the present invention described below for fermentation to produce alcoholic beverages. The target alcoholic beverages include, for example, but not limited to beer, sparkling liquor (happoushu) such as a beer-taste beverage, wine, whisky, sake and the like.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with an decreased level of VDKs, especially DA. Thus, according to the present invention, alcoholic beverages with enhanced flavor can be produced using the existing facility without increasing the cost.

### 5. Yeast Assessment Method of the Invention

The present invention relates to a method for assessing a test yeast for its capability of producing total vicinal diketones or capability of producing total diacetyl by using a primer or a probe designed based on a nucleotide sequence of an acetolactate synthase gene having the nucleotide sequence of SEQ ID NO: 1. General techniques for such assessment method is known and is described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., Methods in Yeast Genetics, Cold Spring Harbor Laboratory Press, 130 (1990)). Using a primer or a probe designed based on a nucleotide sequence (preferably, ORF sequence) of the acetolactate synthase gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the capability of producing total vicinal diketones (VDK) or capability of producing total diacetyl (DA) of the yeast as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the acetolactate synthase gene having the nucleotide sequence of SEQ ID NO: 1 to assess the test yeast for its capability of producing total vicinal diketones (VDK) or capability of producing total diacetyl (DA). In measuring an expression level of the acetolactate synthase gene, the test yeast is cultured and then mRNA or a protein resulting from the gene encoding a protein having a vicinal diketone or diacetyl-reducing activity, is quantified. The quantification of mRNA or protein may be carried out by employing a known technique. For example, mRNA may be quantified, by Northern hybridization or quantitative RT-PCR, while protein may be quantified, for example, by Western blotting (Current Protocols in Molecular Biology, John Wiley & Sons 1994-2003).

Furthermore, test yeasts are cultured and expression levels of the acetolactate synthase gene of the present invention having the nucleotide sequence of SEQ ID NO: 1 are measured to select a test yeast with the gene expression level according to the target capability of producing total vicinal diketones (VDK) or capability of producing total diacetyl (DA), thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the gene having the nucleotide sequence of SEQ ID NO: 1 is measured in each yeast. By selecting a test yeast with the gene expressed higher than that in the reference yeast, a yeast suitable for brewing alcoholic beverages can be selected.

Alternatively, test yeasts are cultured and a yeast with a lower capability of producing total vicinal diketones (VDK) or capability of producing total diacetyl (DA), is selected, thereby selecting a yeast suitable for brewing desired alcoholic beverages.

In these cases, the test yeasts or the reference yeast may be, for example, a yeast introduced with the vector of the invention, a yeast with amplified expression of the gene of the present invention described above, a yeast with suppressed expression of the protein of the present invention described above, an artificially mutated yeast or a naturally mutated yeast. Total amount of vicinal diketones can be quantified by a method, for example, described in Drews et al., Mon. fur Brau., 34, 1966. Total amount of diacetyl can be quantified by a method, for example, described in J Agric Food Chem. 50 (13): 3647-53, 2002. Acetolactate synthase activity can be assessed, for example, by a method of Pang et al. (Biochemistry, 38, 5222-5231 (1999)). The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., Biochemistry Experiments vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954 may be used. Further, whisky yeasts such as Saccharomyces cerevisiae NCYC90; wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Novel Acetolactate Synthase Gene (non-ScILV2)

A novel acetolactate synthase gene (non-ScILV2 gene; SEQ ID NO: 1) specific to a lager brewing yeast was found, as a result of a search utilizing the comparison database described in Japanese Patent Application Laid-Open No. 2004-283169. Based on the acquired nucleotide sequence information, primers non-ScILV2_F (SEQ ID NO: 3) and non-ScILV2_R (SEQ ID NO: 4) were designed to amplify the full-length genes, respectively. PCR was carried out using chromosomal DNA of a genome sequencing strain, Saccharomyces pastorianus Weihenstephan 34/70 strain (also sometimes referred to as “W34/70 strain”), as a template to obtain DNA fragments including the full-length gene of non-ScILV2.

The thus-obtained non-ScILV2 gene fragment was inserted into pCR2.1-TOPO vector (manufactured by Invitrogen Corporation) by TA cloning. The nucleotide sequences of non-ScILV2 gene were analyzed according to Sanger's method (F. Sanger, Science, 214: 1215, 1981) to confirm the nucleotide sequence.

### Example 2

### Analysis of Expression of Non-ScILV2 Gene During Beer Fermentation

A beer fermentation test was conducted using a lager brewing yeast, Saccharomyces pastorianus 34/70 strain and then mRNA extracted from yeast cells during fermentation was analyzed by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

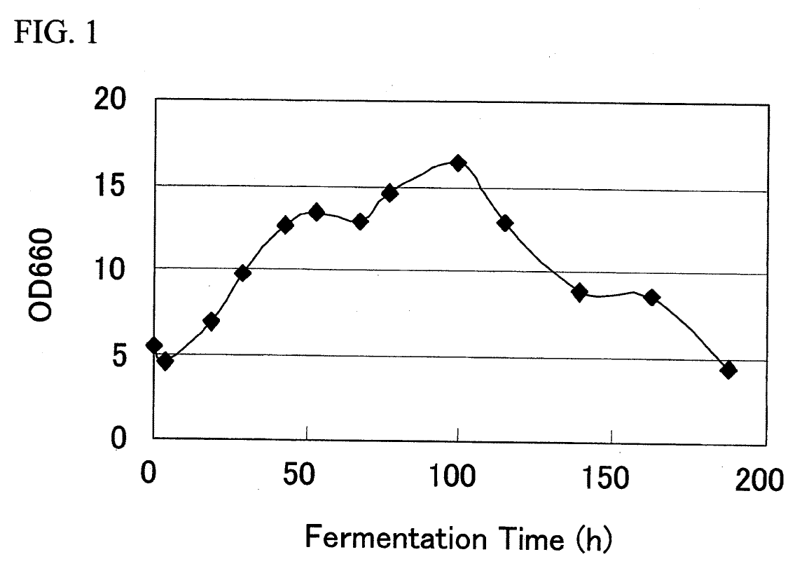

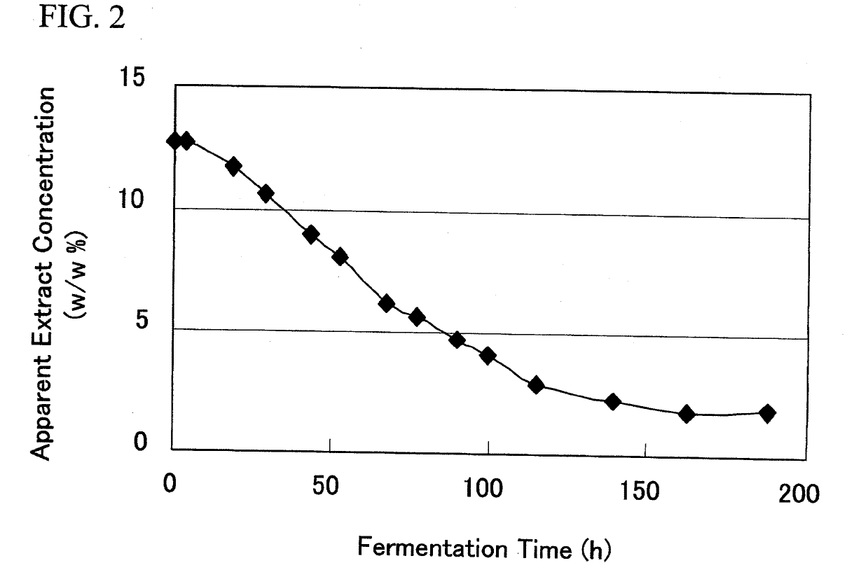

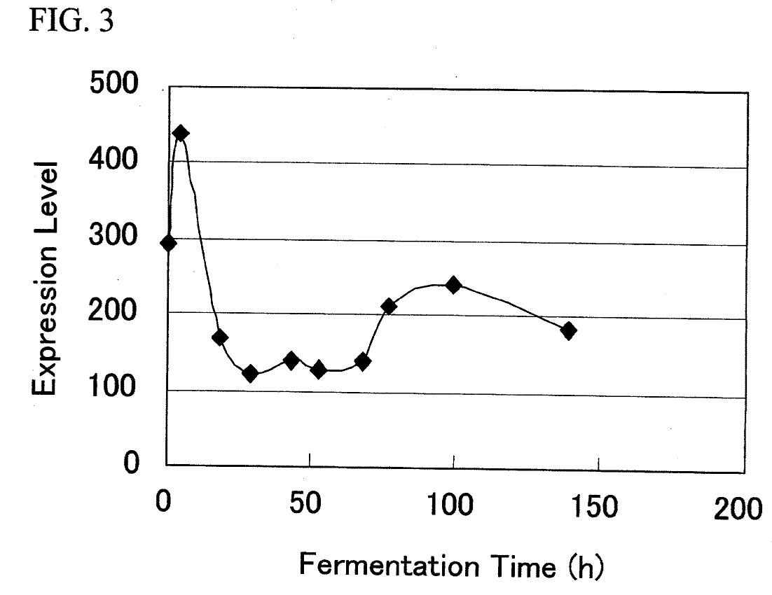

Sampling of fermentation liquid was performed with time, and variation with time of yeast growth amount (FIG. 1) and apparent extract concentration (FIG. 2) was observed. Simultaneously, yeast cells were sampled to prepare mRNA, and the prepared mRNA was labeled with biotin and was hybridized to a beer yeast DNA microarray. The signal was detected using GCOS; GeneChip Operating Software 1.0 (manufactured by Affymetrix Co.). Expression pattern of non-ScILV2 gene is shown in FIG. 3. As a result, it was confirmed that non-ScILV2 gene was expressed in the general beer fermentation.

### Example 3

### Complementation Test of Non-ScILV2 Gene Using Laboratory-Designed Yeast Strain

The function of the product of nonScILV2 gene as an acetolactate synthase was confirmed using a laboratory-designed yeast strain whose endogenous ILV2 gene had been disrupted.

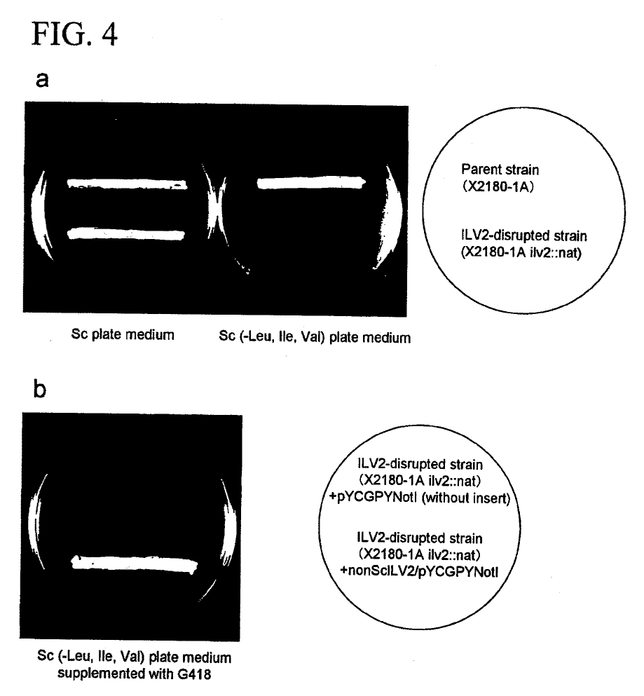

A fragment for disrupting ILV2 gene was prepared by PCR using a plasmid (pAG25 (nat1)) containing a drug resistant marker as a template according to a method described in Coldstein et al., Yeast. 15, 1541 (1999). The sequence of primer utilized are represented by SEQ ID Nos: 5 and 6. S. cerevisiae X2180-1A strain was transformed using the fragment by a method described in Japanese Patent Application Laid-Open No. 07-303475, and selected with YPD plate medium (1% yeast extract, 2% polypeptone, 2% glucose, 2% agar) containing 50 mg/L nourseothricin. Resultant ILV2 gene-disrupted strain was inoculated on SC plate medium without valine, leucine and isoleucine (0.67% yeast nitrogen base without amino acids, 0.2% amino acid mixture (excepting valine, leucine and isoleucine), 2% glucose, 2% agar). Then the strains were cultured for 3 days at 30° C., and the strains were confirmed to be auxotrophic for branched amino acids (FIG. 4-a).

Then, a DNA fragment containing whole coding region of the protein was prepared by digesting the nonScILV2/pCR2.1-TOPO described in Example 1 using restriction enzymes SacI and NotI. This fragment was linked to pYCGPYNot treated with restriction enzymes SacI and NotIA, thereby a nonScILV2 high expression vector nonScILV2/pYCGPYNot was constructed. The pYCGPYNot is a Ycp type yeast expression vector. The introduced gene was highly expressed by the promoter of a pyruvate kinase gene PYK1. A geneticine-resistant gene G418^{r} was included as a selective marker for yeast. Ampicillin-resistant gene Ampr was also included as a selective marker for E. coli.

The resultant high expression vector was transformed to ILV2 gene-disrupted strain (X2180-1A ILV2::nat1). The high expression of nonScILV2 in the transformant was confirmed by RT-PCR A pYCGPYNot introduced strain without insert was prepared as a control. These strains were evaluated in the same way using SC plate medium without valine, leucine and isoleucine. As a result, it was proved that the introduction of nonScILV2 makes the strain non-auxotrophic for branched amino acids (FIG. 4-b, Table 1). That is to say, the product of the nonScILV2 gene was proved to act as acetolactate synthase.

TABLE 1

| | Growth on SC(-Leu, |
|---|---|
| Strain | Ile, Val) plate medium |
| Parent strain (X2180-1A) | Grown |
| ILV2 gene-disrupted strain (X2180-1A ILV2::nat) | Not grown |
| ILV2 gene-disrupted strain (X2180-1A | Not grown |
| ILV2::nat) + pYCGPYNot1 (without insert) | |
| ILV2 gene-disrupted strain (X2180-1A | Grown |
| ILV2::nat) + nonScILV2/pYCGPYNot1 | |

### Example 4

### Disruption of nonScILV2 Gene

Fragments for gene disruption are prepared by PCR using plasmids containing a drug resistance marker (pFA6a(G418r), pAG25(nat1), pAG32(hph)) as templates in accordance with a method described in the literature (Goldstein et al., Yeast, 15, 1541 (1999)). Primers consisting of nonScILV2_delta_for (SEQ ID NO: 7) and nonScILV2_delta_rv (SEQ ID NO: 8) are used for the PCR primers.

A spore clone (W34/70-2) isolated from brewer's yeast Saccharomyces pastorianus strain W34/70 is transformed with the fragments for gene disruption prepared as described above. Transformation is carried out according to the method described in Japanese Patent Application Laid-open No. H07-303475, and transformants are selected on YPD plate medium (1% yeast extract, 2% polypeptone, 2% glucose, 2% agar) containing geneticin at 300 mg/L, nourseothricin at 50 mg/L or hygromycin B at 200 mg/L.

### Example 5

### Analysis of Amount of VDKs Produced During Beer Fermentation

The parent strain and non-ScILV2-disrupted strain obtained in Example 4, are used to carry out fermentation test under the following conditions.

| | |
|---|---|
| Wort extract concentration | 12% |
| Wort content | 1 L |
| Wort dissolved oxygen concentration | approximately 8 ppm |
| Fermentation temperature | 15° C., constant |
| Yeast pitching rate | 5 g wet yeast fungal body/L Wort |

The fermentation broth is sampled with time to observe cell growth (OD660) and extract consumption with time. Quantification of the total VDKs in the fermentation broth is carried out by reacting VDKs (DA and PD) with hydroxylamine to produce glyoxime derivatives, then measuring absorbance of complexes formed from the reaction of resultant glyoxime derivatives and divalent ferric ions (Drews et al., Mon. fur Brau., 34, 1966). The precursors α-acetolactic-acid and α-acetohydroxybutyric-acid are previously converted by a gas washing method (oxidative decarboxylation method) to DA and PD, respectively, to quantify the total VDKs including them.

### INDUSTRIAL APPLICABILITY

According to the method for producing alcoholic beverages of the present invention, because of reduction of the production amount of VDKs, especially DA, which are responsible for off-flavors in products, alcoholic beverages with superior flavor can be readily produced.

1. A polynucleotide selected from the group consisting of:
(a) a polynucleotide comprising a polynucleotide consisting of the nucleotide sequence of SEQ ID NO:1;
(b) a polynucleotide comprising a polynucleotide encoding a protein consisting of the amino acid sequence of SEQ ID NO:2;
(c) a polynucleotide comprising a polynucleotide encoding a protein consisting of the amino acid sequence of SEQ ID NO:2 with one or more amino acids thereof being deleted, substituted, inserted and/or added, and having an acetolactate synthase activity;
(d) a polynucleotide comprising a polynucleotide encoding a protein having an amino acid sequence having 60% or higher identity with the amino acid sequence of SEQ ID NO:2, and having an acetolactate synthase activity;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having an acetolactate synthase activity; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having an acetolactate synthase activity.

2. The polynucleotide of claim 1 selected from the group consisting of:
(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has an acetolactate synthase activity;
(h) a polynucleotide encoding a protein having 90% or higher identity with the amino acid sequence of SEQ ID NO: 2, and having an acetolactate synthase activity; and
(i) a polynucleotide which hybridizes to SEQ ID NO: 1 or which hybridizes to a nucleotide sequence complementary to the nucleotide sequence of SEQ ID NO: 1 under stringent conditions, and which encodes a protein having an acetolactate synthase activity.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A polynucleotide selected from the group consisting of:
(j) a polynucleotide encoding RNA of a nucleotide sequence complementary to a transcript of the polynucleotide (DNA) according to claim 5;
(k) a polynucleotide encoding RNA that represses the expression of the polynucleotide (DNA) according to claim 5 through RNAi effect;
(l) a polynucleotide encoding RNA having an activity of specifically cleaving a transcript of the polynucleotide (DNA) according to claim 5; and
(m) a polynucleotide encoding RNA that represses expression of the polynucleotide (DNA) according to claim 5 through co-suppression effect.

7. A protein encoded by the polynucleotide of any claim 1.

8. A vector comprising the polynucleotide of claim 1.

9. A vector comprising the polynucleotide of claim 6.

10. A yeast, wherein the vector of claim 8 is introduced.

11. The yeast of claim 10, wherein a total vicinal diketone-producing capability or diacetyl-producing capability is reduced by introducing the vector comprising the polynucleotide.

12. The yeast of claim 11, wherein a total vicinal diketone-producing capability or total diacetyl-producing capability is reduced by decreasing an expression level of the protein encoded by the polynucleotide.

13. A yeast, wherein an expression level of the polynucleotide (DNA) of claim 5 is repressed by introducing the vector comprising the polynucleotide, or by disrupting a gene related to the polynucleotide (DNA) of claim 5.

14. A method for producing an alcoholic beverage comprising culturing the yeast of claim 10.

15. The method for producing an alcoholic beverage of claim 14, wherein the brewed alcoholic beverage is a malt beverage.

16. The method for producing an alcoholic beverage of claim 14, wherein the brewed alcoholic beverage is wine.

17. An alcoholic beverage produced by the method of claim 14.

18. A method for assessing a test yeast for its total vicinal diketone-producing capability or total diacetyl-producing capability, comprising using a primer or a probe designed based on a nucleotide sequence of an acetolactate synthase gene having the nucleotide sequence of SEQ ID NO: 1.

19. A method for assessing a test yeast for its total vicinal diketone-producing capability or total diacetyl-producing capability, comprising: culturing a test yeast; and measuring an expression level of an acetolactate synthase gene having the nucleotide sequence of SEQ ID NO: 1.

20. A method for selecting a yeast, comprising: culturing test yeasts; quantifying the protein according to claim 7 or measuring an expression level of an acetolactate synthase gene having the nucleotide sequence of SEQ ID NO: 1; and selecting a test yeast having said protein amount or said gene expression level according to a target total vicinal diketone-producing capability or total diacetyl-producing capability.

21. The method for selecting a yeast according to claim 20, comprising: culturing a reference yeast and test yeasts; measuring an expression level of an acetolactate synthase gene having the nucleotide sequence of SEQ ID NO: 1 in each yeast; and selecting a test yeast having the gene expressed lower than that in the reference yeast.

22. The method for selecting a yeast according to claim 20, comprising: culturing a reference yeast and test yeasts; quantifying the protein encoded by the polynucleotide in each yeast; and selecting a test yeast having said protein for a smaller amount than that in the reference yeast.

23. A method for producing an alcoholic beverage comprising: conducting fermentation for producing an alcoholic beverage using the yeast according to claim 10 or a yeast selected by the method according to claim 20; and adjusting the total vicinal diketone-producing capability or total diacetyl-producing capability.
